(19) United States
(12) Patent Application Publication
(10) Pub. No.: US 2009/0030454 A1
(43) Pub. Date: Jan. 29, 2009

(54) RESORBABLE IMPLANTABLE DEVICES

(76) Inventors: David Philip Knight, Winchester (GB); Christine Ortlepp, Berks (GB); Nicholas Skaer, London (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/910,949

(22) PCT Filed: Apr. 5, 2006
(86) PCT No.: PCT/EP06/03471

§ 371 Date: Sep. 25, 2008

(30) Foreign Application Priority Data
Apr. 8, 2005 (GB) 0507161.8
Oct. 13, 2005 (GB) 0520828.5

(51) Int. Cl. A61L 17/00; C07K 14/78; A61F 2/08; A61B 17/04

(52) U.S. Cl. 606/230; 530/353; 606/231; 623/13.18

(57) ABSTRACT

A resorbable implantable device, such as a suture or prosthesis, comprising one or more silk elements made at least partially (poly)alanine Wild silk proteins of a saturnid moth or analogues thereof.

### TECHNICAL FIELD

This invention relates to resorbable implantable devices based on a poly(alanine) Wild silk and to methods for producing such devices and the materials from which they are formed. In particular, one embodiment of the invention provides a resorbable suture.

### BACKGROUND ART

The ability to make resorbable materials and devices for implantation in human or animal bodies can confer considerable advantages. First the implanted material or device breaks down slowly in the body and is slowly replaced by natural tissue whose functionality and mechanical integration into the healing tissue is likely to be superior to that of the implanted material, reducing the risk of eventual mechanical failure, a serious problem with non-resorbable orthopaedic implants. Secondly, the properties of synthetic non-resorbable materials and the long time these are often required to remain in contact with tissues result in increased risk adverse reactions. Thirdly, they avoid the risk of morbidity, pain and risk of infection associated with a further surgical procedure required to move failed or otherwise unsatisfactory non-resorbable material or devices. A biocompatible and resorbable fibrous material would thus be highly advantageous for a wide range of implantable materials and devices of which resorbable sutures are the simplest.

The use of animal gut as a resorbable suture for wound closure has been known since ancient times (Galen 175AD). Sterile resorbable sutures prepared from animal gut were introduced by B. Braun Medical Inc in 1909. A wide range of potentially resorbable suture materials, including gut, leather, horse hair, were used until synthetic resorbable sutures were produced in consequence of advances in polymer chemistry in the 1960's. The best natural absorbable sutures were originally prepared from ‘catgut’, submucosal or mucosal collagen derived from ovine intestines or bovine intestines. Concerns about the transmission of diseases and adverse inflammatory responses have lead to recommendations for the discontinuation of their use.

The first synthetic resorbable sutures were constructed from polyglycolic acid or polyglactin 9. A range of synthetic resorbable sutures were subsequently developed including: Dexon® (Poly(glycolic acid); Vicryl® (90/10 Poly(glycolide-L-lactide)); Panacryl (5/95 Poly(glycolide-L-lactide); USSC C0's Glycolide-trimethylene carbonate copolymer; PDS II® (Poly-p-dioxanone); Maxon® (Poly(glycolide-co-trimethylene carbonate)); Monocryl® (Poly(glycolide-caprolactone copolymer)); Biosyn® (Glycolide-trimethylene carbonate copolymer); Monosyn® (Glycolide-trimethylene carbonate-caprolactone tri-composite copolymer); and a Lactide-caprolactone copolymer.

Resorbable sutures are generally considered to have the following advantages compared with non-resorbable sutures:
1. The use of non-resorbable materials is usually accompanied by encapsulation or immune rejection. Although the resorption process for resorbable materials is always associated with some inflammatory reaction within the tissue surrounding the material, resorption is thought to reduce the length and intensity of tissue exposure to physical, chemical and immunogenic irritants compared to non-resorbable sutures. It is agreed that in general this results in a reduction in adverse tissue reaction in resorbable sutures compared with non-resorbable ones.
2. Resorbable materials are generally said to result in lower suture infection rates than non-resorbable materials, although this is not always the case.
3. The use of resorbable sutures reduces the discomfort, risk of infection and costs involved in procedures incurred by the additional procedure required to remove non-resorbable sutures or other methods of wound closure.
4. The use of resorbable sutures (gut and Vicryl) in periodontal surgery are said to attract less placque formation than non-resorbable sutures.

Synthetic resorbable sutures are said to offer the following advantages over collagenous resorbable sutures:
1. Mechanical properties are usually better and the standardized manufacturing process results in greater batch-to-batch consistency.
2. Unpredictability of degradation of catgut is a problem.
3. Degradation rates are slower for polyglycolic acid compared with catgut.
4. There is general agreement that synthetic sutures produces less tissue reaction than catgut.
5. Degradation of catgut occurs is faster in infected sites while synthetic sutures are far less affected.
6. Catgut sutures appear to be associated with a greater risk of infection.
7. Collagenous sutures have a potential, but to date unquantified, risk of causing prion disease.
8. Delayed resorption as a consequence of radiotherapy may provide time for calcifications on collagenous suture material. These may be a nuisance in mammographic examinations.
9. Plain and chromic catgut sutures rapidly disintegrate in human gastric juice, bile, pancreatic juice, and mixtures of these making them unsuitable for many gastrointestinal uses. Mulberry silk sutures do not suffer from this disadvantage.

Possible disadvantages of synthetic resorbable suture materials over collagenous resorbable sutures are:
1. Subjective evidence of reduced knot strength and greater slippage.
2. Braided synthetic sutures may have a greater tendency to tear through oedematous or fragile tissues or parenchymatous organs and may be worse at cutting surgeons' gloves.
3. They may have significant toxicities.
4. Of a range of suture materials studied, poly L-lactide/glycolide showed the highest incidence of granulation tissue and infection while another long resorption time suture material, polydioxanone also resulted in some granulation tissue and a significant infection rate.

Resorbable sutures whether prepared from natural proteins or synthetic polymers are broken down by enzymatic hydrolysis. Partially hydrolysed fragments are then taken up by phagocytosis and eventually degraded to monomeric units within phagosomes. Thus the resorption rate of resorbable sutures is thought to depend largely on the rate at which they are broken down by enzymatic hydrolysis in the tissues. The resorption rate is best quantified as the half-resorption time, i.e. the time required to reduce the ultimate tensile strength of a suture material by 50%. This property is measured by in vivo testing of the device in question using a representative sample an appropriate host and an appropriate location in the host, depending on the intended use of the device.

Half-resorption time is an important parameter in the design of resorbable sutures; it is highly desirable that there is an approximate match between the rate of decline of the tensile strength of the suture due to resorption and the rate of increase in tissue strength of the wound or anastomoses due to wound healing. A similar match between resorption rate and rate of tissue healing is also required in the case of prostheses substantially comprised of resorbable materials. The rate of increase in tensile strength of a sutured wound, incision or anastomoses will in turn depend on the location and size of the wound and the healing rate. The rate of healing of the wound will depend in turn on a range of factors including physiological condition, extent of inflammation, the presence and severity of infection, nutritional statues, gender, age etc. Thus the half-resorption time for a given suture will define the applications for which sutures can be used.

The range of half-resorption times in synthetic polymers used for sutures depends largely on the crystallinity of the polymer. The more compact crystalline structures of monobloc polymers such as poly-p-dioxanone or that of Panacryl (which is dominated by Poly(lactide) exclude both hydrolytic enzymes and water delaying hydrolysis. Thus these suture materials have half-resorption times of about 6 months. On the other hand the copolymers such as Glycolide-trimethylene carbonate-caprolactone tri-composite copolymer Poly(glycolide-co-trimethylene carbonate) lack crystalline regions and consequently have much shorter half-resorption times of the order of two weeks. The molecular weight, hydrophobicity and morphology of the synthetic polymer will also affect the rate of hydrolysis: polymers with a larger molecular weight generally take longer to resorb than polymers with a smaller molecular weight. Polymers with higher hydrophobicities resist swelling in water and this leads to reduced ingress of hydrolytic enzymes, thus slowing resorption times. Fibres with smaller diameters have larger surface to volume ratios and tend to be resorbed more rapidly than fibres with larger diameters.

Silk derived from the cocoon of the Mulberry Silkworm, Bombyx mori has been used for many years as a non-resorbable suture. In contrast, dragline silk of the spider Nephila clavipes appears to be rapidly resorbed within 15 days after being implanted into the dermis of pigs as shown in VOLLRATH, F., et al. Local tolerance to spider silks and protein polymers in vivo. In Vivo. 2002, vol. 16, p. 229-234. The much more rapid resorption of spider dragline silk compared with mulberry silk may stem from the considerably lower crystallinity of the spider dragline silk compared with the mulberry silk. The lower hydrophobicity of spider dragline protein may also contribute to its rapid resorption as shown by our examination of the published sequence of Spidroin I, the principle structural component of Nephila spider dragline. This shows that the hydrophilicity of the repetitive central part of Spidroin I is considerably less than that of the repetitive region of Bombyx mori heavy chain fibroin, the main structural component of mulberry silk.

Two general methods influencing the resorption rate of resorbable sutures have been described in the patent and scientific literature: the application of a hydrophobic coat to delay the entry of hydrolytic enzymes; and inclusion in the silk of transition elements, such as chromium, nickel or cobalt. The transition elements may delay resorption by inhibiting phagocytosis or may reduce the release of proteolytic enzymes capable of hydrolyzing the silk by reducing the number of white blood cells in the vicinity of the suture material. A potential disadvantage of the latter approach is that these transition metals are potentially carcinogenic. Furthermore the chromium in chromic catgut may produce some neuropathy.

An example of the use of a hydrophobic coating material to delay the resorption of a resorbable suture is disclosed in U.S. Pat. No. 6,616,687 (GUNZE KK) Sep. 9, 2003 which teaches a hydrophobic coating of caprolactone and lactide and calcium stearate. Similarly a paper by D. Bichon, W. Borloz, and A. L. Cassano-Zoppi, entitled ‘In vivo evaluation of a new polyurethane-coated catgut suture’ which appeared in Biomaterials, 5, 255, 1984 teaches the use of a polyurethane coating to delay resorption of a collagenous suture. U.S. Pat. No. 4,364,393 teaches the addition to a monocarboxycellulose suture of a transition element taken from the group consisting of Fe, Ni, Co, Bi, Mn or a combination of these to enable its disintegration time to be matched to the wound healing time.

Thus there is considerable scope for improvement in the performance of resorbable sutures. While Panacryl® and PDS have the best resorption rates and have good mechanical strength, Panacryl has been withdrawn by its manufacturers Ethicon while PDS is associated with high infection rates and unwanted tissue reactions. New methods and materials are required to produce resorbable suture materials with reduced toxicity, tunable resorption and low infection rates.

International Patent Application WO 01/56626 (Nexia Biotechnologies) teaches the use of a surgical suture comprising spider silk or a composite containing spider silk and a non-spider silk structural element wherein the said non-spider silk element is hyaluronic acid, elastin or collagen.

International Patent WO 2004/016651 (The University of York) teaches the preparation by genetic engineering of a polypeptide containing the sequence motive AGRGQGGYGQAAG derived from an arachnid and at least two motifs rich in polyalanine wherein the said polyalanine motifs comprise at least 6 alanine amino residues. WO 2004/016651 also teaches other variant polypeptide sequences based on the above sequence motifs. It also teaches the use of silk polypeptides based on these sequences for the manufacture of material.

United States Patent Applications 20040102614 and 20050054830 teach a method of spinning recombinant spider silk or recombinant silkworm proteins whose sequence is derived from insects such a Bombyx mori. It also teaches a variety of uses for the silk filaments including a medical suture, skin graft substitute, replacement ligament, medical adhesive strip, surgical mesh.

U.S. Pat. No. 4,818,291 teaches the use of a liquid adhesive which contains a mixture of human-fibrinogen and silk-fibroin and which is suitable for use in surgery. The silk-fibroin is obtained from domestic silk or a Wild silk, or a mixture thereof. This patent pertains solely to a surgical adhesive to act as a filler for adherend sutured surfaces.

Tsukada, M., Arai, T., Colonna, G. M., Boschi, A., Freddi, G. in their paper “Preparation of metal-containing protein fibers and their antimicrobial properties” which appeared in Journal of Applied Polymer Science 2003 89, 638-644, teach that the binding of transition element ions Ag+, Cu2+, Co2+ to Bombyx mori and Antheraea pernyii silk after derivitisation to introduce chelating groups resulted in significant antimicrobial properties.

Brunet, P. C. J and Coles B. in their article entitled “Tanned Silk” published in Proc. Roy. Soc. Ser B. 187, 133-170 in 1974 described the existence of a peroxidase-based tanning system in Antheraea pernyii silk. The occurrence of dityrosine in the silk of Antheraea pernyii has been noted by Raven, J., Earland, C., and Little, M., in their article entitled “Occurrence of dityrosine in Tussah silk fibroin and keratin” in Biochim. Biophys. Acta 251, 96-99 published in 1971. It is likely that the dityrosine identified by the latter Raven et al arises from the action of the bound peroxidase described by the Brunet et al on the tyrosine residues present in the heavy chain fibroins of Antheraea pernyii, yamamai, and myllita.

The use of heating in a solution containing citrate ions as a non-toxic method of toughening mulberry silk cloth is taught by Leksophee, T., Supansomboon, S. and Sombatsompop, N. in their paper entitled “Effects of crosslinking agents, dyeing temperature, and pH on mechanical performance and whiteness of silk fabric” which appeared in Journal of Applied Polymer Science 91 (2) 1000-1007 (2004). Formic acid has been rather widely used as a solvent for silk proteins and silk-like peptides. The following papers disclose that the use of formic acid promotes the formation of β-sheets when protein films or electrospun fibres are prepared from solutions of silk proteins or peptides dissolved in formic acid: Asakura, T., Tanaka, C., Yang, M. Y., Yao, J. M. and Kurokawa, M. “Production and characterization of a silk-like hybrid protein, based on the polyalanine region of Samia cynthia ricini silk fibroin and a cell adhesive region derived from fibronectin” Biomaterials 25 (4) 617-624 (2004).

The use of hot dilute tartaric acid solution as a means of degumming silk has been described by Chopra, S. and Guiraiani, M. L. in their article entitled “Comparative evaluation of the various methods of degumming silk” which appeared in Indian Journal of Fibre and Textile Research 19 76-83 (1994).

L. Kreplak, J. Doucet, P. Dumas, and F. Briki in their article entitled “New Aspects of the α-Helix to β-Sheet Transition in Stretched Hard α-Keratin Fibers” which appeared in Biophysical Journal Volume 87 Jul. 2004 640-647 teach that slowly stretching the keratin fibers of horse hair to 40-60% of their resting length in water or at humidities in excess of 30% produced an increase in increase in crystalline β-Sheet component while similar effects could be produced by slowly stretching horse hairs to 100% of their length and then treating them with steam at 100° C. for one hour.

The object of the invention is to provide resorbable devices such as sutures which substantially avoid some or all of the disadvantages inherent in existing resorbable sutures discussed above. The use of Wild silk is proposed to allow the objects of the invention to be achieved.

### DISCLOSURE OF THE INVENTION

One aspect of the invention comprises an implantable device comprising a structure formed from one or more silk elements made at least partially of (poly)alanine Wild silk protein.

Particularly preferred forms of the implantable device are a suture or a prosthesis.

The use of (poly)alanine Wild silk provides a material that is resorbable by the body and has the ability to tune the resorption rate of the device while retaining good mechanical properties.

The term “implantable device” refers to a device implanted into a human or animal body during surgery and the term “Wild silk” refers to silk produced by saturniid caterpillars. The saturniid species Antheraea pernyi, Antheraea yamamai, Antheraea militta, Antheraea assama, Philosamia Cynthia ricini and Philosamia Cynthia pryeri are currently used for the commercial production of Wild silk. For the purpose of this application, the term “biocompatible” has the meaning that the material does not induce an adverse cellular reaction in mammalian cells. This can be assessed by a variety of in vitro methods including: the inability to kill mammalian cells in cell culture; the lack of induction of cytokine release from living mammalian cells; and the lack of a retarding effect on cell proliferation in culture.

It is furthermore an object of the invention to provide a material for the implantable device whose strength and toughness is good and whose resorption rate can be tuned to give half-resorption times ranging from 2 weeks to 9 months.

The silk elements can be either derived from the cocoons of the Wild silk moth or they are formed from a solution of regenerated saturniid silk. Their half-resorption time can be controlled to vary from a period of 2 weeks to 9 months which allows the implantable device to be adapted to the natural healing rate of any tissue.

In one advantageous embodiment of the invention, a peroxidase is bound to the silk protein. The peroxidase has anti-bacterial properties which aids the healing of the wound.

The silk elements can be formed from a solution of regenerated silk.

A silk protein matrix can be provided between the silk elements.

Preferable the implantable device is manufactured using a silk protein matrix between the one or more silk elements. The protein matrix can be wholly or partially comprised of native or regenerated silk protein derived from one or more species of saturnid or bombycid silk worm.

The invention also provides a method for the manufacture of an implantable device. In a first step (poly)alanine Wild silk is provided and in a second silk elements are manufactured from the silk protein. In one embodiment, the silk elements are extruded.

The step of providing poly(alanine) Wild silk protein can comprises unreeling silk from a cocoon of a saturniid Wild silk moth. Alternatively, a native silk protein solution can be obtained directly from a poly(alanine) Wild silkworm.

The poly(alanine) Wild silk protein can also be provided by the dissolution of a degummed silk to form a regenerated silk solution.

Preferably, the method further comprises varying the degree of crystallinity or cross-linking of the silk element, for example by exposing the silk element to formic acid, methanol, hot di- or tricarboxylic acid (e.g. citric acid), or a dialdehyde (e.g. glutaraldehyde), or combinations of one or more of these.

The method can also comprise exposing the silk elements to steam and stretching them.

In one preferred embodiment, the silk elements are exposed to a reducing agent after exposing them to a dialdehyde.

The silk elements can also be exposed to a solution containing one or more amino acids after exposing them to a dialdehyde and before exposing them to a reducing agent. The reducing agent can be sodium borohydride or cyanoborohydride.

The method can also comprise binding peroxidase to the silk element.

Another embodiment comprises exposing the silk element to a methanol solution of between 30% and 60% by volume.

The implantable device can be made into a suture, a prosthesis, or other medical devices.

### BRIEF DESCRIPTION OF THE DRAWINGS

FIG. 1 shows a typical stress/strain plot of the silk fibre prepared according to this invention.

### MODE(S) FOR CARRYING OUT THE INVENTION

Two typical examples of an implantable device according to the invention comprise a suture and an ankle ligament prosthesis. Both implantable devices are comprised wholly or partially from one or more silk filaments as will be described below. The silk filaments comprise polyalanine silk proteins.

An implantable device, such as a suture, according to the invention is comprised wholly or partially from one or more silk filaments. The silk filaments can be obtained in a number of ways from Wild silk moths. In a first way the silk filament is obtained directly by unwinding the silk filaments from the cocoons of a saturniid moth. The saturniid moths may come from the genera Antheraea, Anaphe, Philosamia, Perisomena, Atacus, Cricula, Archaeatacus, Saturnia, Caligula, Loepa, Copiopteryx, Colaradia, Hemileuca, Memareia, Opodiphthera, Coscinocera, Gonometa, Pseudohazis, Rothschildia, Anisota, Actias, Io, Bunaea, Henochia, Pseudobunae, Lepa, Argena, Coscinocera, Cecropi, or Gynanisa.

In a second way the silk filament is spun from a solution of the silk protein (fibroin) of a saturniid moth. The spinning of a silk filament from a solution of a silk protein is known in the art, for example, in the International Patent Application No WO 01/38614. The fibroin is obtained either directly from the silkworms or a regenerated silk solution. The regenerated silk solution is prepared by dissolving silk in one or more chaotropic agents (e.g. 11.6 molar Lithium bromide).

Many species of saturniid moth pupae are very large compared with those of other subfamilies and often remain at the pupal stage for prolonged periods. They are therefore a potentially valuable food source to other organisms. The possession of chemical defense mechanisms in the cocoon silk to prevent attack from birds, fungi, bacteria and other organisms has large selective advantage. Thus certain saturniid silks contain a bound peroxidase and tyrosinase which may have an antimicrobial effect as well as acting as a cross-linking agent.

The saturniid silk preferably contains a bound peroxidase. Thus its use in an implantable device may provide long lasting broad spectrum bactericidal action by the production of free-radicals when implanted into human or animal tissues. In one embodiment of the invention human or recombinant human peroxidase can be artificially bound to the silk in order to improve the anti-microbial properties of the silk elements and thus the implantable device.

In one embodiment of the invention, a suture is created by twisting, braiding or plaiting the degummed silk filaments obtained as described above. The preferred embodiment uses degummed silk prepared from a raw silk comprising 4 to 10 silk baves slightly twisted together, each bave reeled from one cocoon and comprising two silk filaments (brins). In the preferred embodiment, the brins have a flattened ribbon shape with minor thickness 10.2±2.2 μm and major thickness 31.9±4.2 μm. The suture can also be a monofilament extruded or otherwise formed from a saturniid silk protein or from an analogue of the saturniid silk protein. Fine double stranded filaments can be obtained by reeling a single bave from a single cocoon. Both the monofilaments and the fine double stranded filaments can be used directly and are useful for providing extremely fine and tough filaments for use in microsurgery or for incorporation into other implantable devices. In one embodiment silk filaments can be wound on to a former to form fibre lays for use in implantable device. These fibre lays can be used directly or can be stitched through during winding or after completion of the winding of the fibre lay. In a further embodiment weaving of filaments or twists or plaits or braids can be used to produce fibre lays for use in implantable devices.

The silk can be strengthened and toughened by chemical treatment before or after incorporation of the silk filament into the suture or other implantable devices. Three methods of chemical treatment have been found to be efficacious. The first method comprises treatment with solutions of glutaraldehyde or other di-aldehydes at 3-50° C.; the second, treatment with a hot di- or tri- or poly-carboxylic acid, such as citric acid; and third, treatment with cold formic acid and or methanol. The latter treatment is designed to increase the beta sheet content of the silk protein and thus the crystallinity of the protein. It is to be noted that increasing the crystallinity of the protein in this way increases the resorption time of the silk within the suture or implantable device.

Where glutaraldehyde or another dialdehyde is used as a treatment to strengthen and toughen the silk the resulting aldimine (Schiff base) crosslinks between the aldehyde groups and the free amino groups of the protein are relatively weak, tending to hydrolyse with time or with mechanical strain. This results in the rupture of cross-links and the release of free aldehyde groups which are potentially toxic. In addition toxic aldehyde groups are introduced into proteins by the failure of one of the aldehyde groups of the dialdehyde to react with the protein. The weakness of the aldimine crosslink and the problem of toxicity of aldehyde groups introduced by dialdehyde treatment can be overcome by treating the silk with a suitable reducing agent after treatment with the dialdehyde dilute. One preferred reducing agent comprises an aqueous sodium borohydride solution. This chemically reduces the weak aldimine cross-link into a much more stable secondary amine link and reduces free aldehyde groups to the corresponding non-toxic alcohols. It may be advantageous to react the free aldehyde groups with the primary amino group of an amino acid such as glycine, valine or lysine before reduction. If an amino acid is used in this way either cyanoborohydride or sodium borohydride can be used as the reducing agent. It will be understood that the selection of the amino acid can be used to tune the hydrophobicity of the silk. Thus, for example, the reaction of a free aldehyde group with lysine will result in the addition a free amino and carboxyl group while reaction of a free aldehyde group with glycine will result in the addition of only a free carboxyl group.

Treatment with a borohydride or cyanoborohydride after glutaraldehyde therefore wholly or substantially removes the toxic effect resulting from aldehyde cross-linking. It is also likely that treatment with a borohydride with or without pre-treatment with a selected amino acid can be used to modify the resorption time of the silk in a suture or implantable device.

The treatment with borohydride can be applied to any silk material (i.e. is not only restricted to Wild silk) to reduce the toxic effects resulting from aldehyde cross-linking arising from the described chemical treatments.

The suture or implantable device can be made more hydrophobic by treating the silk protein with alkylating agents as described UK patent application 0516846.3. Increasing the hydrophobicity in this way increases the resorption time of the silk within the suture or implantable device.

Thus the resorption time of the silk within the suture or implantable device can be controlled by varying three factors: the hydrophobicity; the extent and nature of the covalent cross-linking; and the extent of β-sheet crystallinity.

Other filaments or fillers can be added to the silk filaments made from saturniid silk or from the saturniid silk analogue to form the suture. In one embodiment, regenerated silk protein prepared by dissolving the silk of one or more saturniid or a bombycid or a combination of these can be used to strengthen and toughen the sutures or improve handling by providing a regenerated silk protein matrix between the silk filaments of the suture. The regenerated silk protein matrix can be rendered insoluble by treatment with an aldehyde, by cold formic acid vapour or by methanol.

In addition, a range of fillers, coatings or dressings can be applied to silk sutures and other implantable devices to improve their properties.

### EXAMPLE 1

### Increase in the Strength and Toughness of Sutures by Treating with Hot Citric Acid

The tensile properties of the silk filaments and the sutures prepared from the silk filaments can be improved significantly by treatment of the silk filaments with hot citric acid as follows: A 5% solution of citric acid was prepared and adjusted to pH 5.5 with concentrated and dilute sodium hydroxide solution. The silk filaments were treated in the citric acid solution for one hour at 80 degrees centigrade. After removal from the citric acid solution, the silk filaments were washed thoroughly in distilled water (3×30 minute) and then allowed to dry in air.

This treatment produces an increase in ultimate tensile strength of approximately 5% and a 10-15% increase in extensibility as measured as described below on an Instron mechanical testing instrument.

Consequently the treatment produces a considerable increase in toughness of the silk filament and also of the suture made from the silk filament.

### EXAMPLE 2

### Extension of the Resorption Time of the Suture

The resorption time of the silk filament of the invention is increased by treating them with formic acid and methanol before or after the addition of silk protein filler as follows: The silk filaments (or sutures made from the silk filaments) are immersed in a 10-95% formic acid solution or exposed to the vapour from the formic acid solution for 1 to 5 hours at room temperature. The silk filaments are washed and allowed to dry in air. This treatment results in an increase in the degree of crystallinity of the silk filaments, as is determined by X-ray crystallography or by measuring the glass transition temperature of the completely dry fibre by differential scanning calorimetry measurements.

An additional increase in crystallinity can be achieved by additionally immersing the silk filaments in 30-100% methanol solution or exposing the silk filaments vapour from this solution for 1 hour. The silk filaments are washed and allowed to dry in air. Good results are achieved at 40% methanol solution.

These treatments increase the crystallinity and extend the resorption times for the silk filaments. They also render insoluble the coatings derived from aqueous silk solutions.

### EXAMPLE 2a

In an alternative embodiment the silk filaments (or suture) are, prior to treatment with the formic acid solution, painted or immersed in regenerated bombycid or saturniid regenerated silk solution.

### EXAMPLE 3

### Conjugation of Peroxidase to Silk Proteins

1. 16 mg horse radish peroxidase (HRP) was dissolved in 4 ml of deionised water and 0.8 ml of sodium periodate. The mixture was stirred for 20 minutes at room temperature. 6 drops ethylene glycol were added to the mixture which was then stirred for a further five minutes at room temperature to form an oxidized HRP solution.
2. 25 mg of silk filament were soaked in distilled water for 30 minutes and the pH adjusted to 9.0-9.5 with 1M sodium carbonate solution. The oxidized HRP solution was added and the resultant solution stirred for 2 hours at room temperature.
3. 0.4 ml of freshly prepared 0.4% w/v sodium tetraborate solution was added to the resultant solution and the silk elements were incubated in the solution for 2 hours at 2-8° C.
4. Finally the silk elements were washed thoroughly and air dried.

The effectiveness of the conjugation reaction can be assessed by staining with the diaminobenzidine method for peroxidase. An unconjugated control is necessary as saturniid silks have appreciable endogenous peroxidase activity.

Other methods of conjugating peroxidase to silk fibres will be understood by persons skilled in the art. In a further embodiment a reduction step with mercaptoethanol followed by an oxidation step can be used to couple peroxidases to the cysteines in the N- and C-terminii of poly(alanine) Wild silk fibroins.

Protocols for demonstrating the effects of reagents on the tensile properties of Saturniid silks.

a. Treatment with Formic Acid

Raw silk (7 bave silk) was wound onto glass slides under slight tension. An epoxy glue was applied to both ends of the slide and allowed to harden to hold the silk in place. The slide was placed in a 500 ml sealed jar containing an atmosphere saturated with 98% formic acid. Samples were not in direct contact with the formic acid for 60 minutes at room temperature and then removed and rinsed well with deionised water.

b. Treatment with Formic Acid and Methanol.

Samples were immersed in absolute methanol for a further 60 min at room temperature after treatment with formic acid.

c. Treatment with Citric Acid

Samples were immersed for 1 hour at 80° C. in aqueous 5% w/v citric acid adjusted to pH 5.5 using sodium hydroxide and washed thoroughly with distilled water.

d. Mechanical Testing.

Samples were mounted without preload in cardboard mounts. All samples were soaked for one hour at room temperature in distilled water to ensure full hydration and were tested to failure at 100% relative humidity at room temperature on an Instron Universal Materials Testing Instrument at a strain rate 50%/min using a 1 Newton load cell. As reliable cross-sectional areas are not available at present and all samples came from the same skein, a normalized cross-sectional area of 7854 μm² was used throughout thus all reported values are nominal. Stress strain curves were plotted using Excel. Sigma stat 3.1 was used for statistical analysis. Other comparisons were made with One Way ANOVA, with multiple comparisons vs control group using the Holm-Sidak method.

Table 1. Mechanical properties of treated and untreated Antheraea pernyi 14 brin silk fibres presented as average values±standard deviation. Results that are significantly larger than those of the control are indicated as follows: * p=<0.01; ** p=<0.001; *** p<0.0001; **** p<0.00001; *****p<0.000001

TABLE 1
Table 1. Mechanical properties of treated and untreated Antheraea pernyi 14
brin silk fibres presented as average values ± standard deviation.

| | | | Ultimate Tensile Stress | Toughness |
|---|---|---|---|---|
| Treatment | n | Strain (%) | (GPa) | kJkg-1 |
| control | 27 | 31.6 ± 6.4 | 0.421 ± 0.041 | 67 ± 16 |
| citric acid | 12 | 35.2 ± 3.3 | 0.436 ± 0.035 * | 76 ± 12 |
| formic acid | 16 | 33.5 ± 4.9 | 0.464 ± 0.040 ****** | 83 ± 17 *** |
| 40% methanol | 13 | 35.2 ± 4.5 | 0.447 ± 0.030 | 81 ± 11 * |
| 100% methanol | 29 | 34.4 ± 6.1 | 0.375 ± 0.047 | 63 ± 16 |
| formic acid + | 16 | 37.1 ± 4.2 ** | 0.484 ± 0.023 **** | 89 ± 13 **** |
| 100% methanol | | | | |

Results that are significantly larger than those of the control are indicated as follows:
* p = <0.01;
** p = <0.001;
*** p < 0.0001;
**** p < 0.00001;
***** p < 0.000001

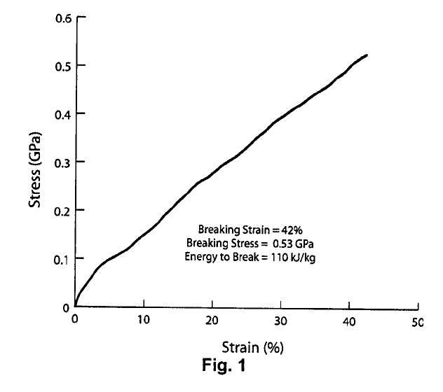

A typical stress/strain plot of formic acid/100% methanol treated 14 brin silk fibre tested wet is shown in FIG. 1.

These results show that the combined treatment with formic acid and methanol produced an average increases in strain to failure of 21%, ultimate tensile strength of 18% and toughness of 57% compared with untreated controls. Compared with Nephila edulis spider dragline silk as a benchmark, the enhanced Wild silk fibres have a similar average strain to failure and a good ultimate tensile strength 40% of that of spider dragline silk and therefore more than twice the tensile strength of high tensile steel weight for weight. The typical fibre shown in FIG. 1 has a toughness of 70% of that of dry Nephila edulis spider dragline silk. As both formic acid and methanol are known to increase the increased β-sheet content, these results strongly suggest that in the formation of this conformation in fibroin is incomplete in the silk as spun by the silkworm and that further intermolecular β-sheet formation with an increase in crystallinity can be produced by appropriate treatment. In addition, three possible mechanisms may account for the effect of the citric acid treatment on the ultimate tensile of the silk: 1. Increased β-sheet content produced by heating; 2. Increased β-sheet content produced by the citric acid in an analogous way to that produced by the formic acid; 3. Formation of intermolecular lysine-citrate-lysine di-amide cross-links as suggested by Leksophee et al., 2004 (see above).

Taken together the above results strongly suggest that the tensile properties of silks can be improved and tuned by appropriate physico-chemical treatments designed to increase the β-sheet content. Further, as degree of crystallinity has an important determinant of resorption times of resorbable suture materials, these observations strongly suggests that the resorption times of Wild silks can be tuned by appropriate treatments that modulate the degree of crystallinity.

### EXAMPLE 4

### The Preparation of a Prototype Ligament Prosthesis and Implantable Sheet Materials from Wild Silk Filaments Treated with Formic Acid and Methanol

1. Degummed Wild silk was treated with formic acid and methanol as described in protocol 1B above. After washing and drying the silk fibres were wound with a crossing angle of approximately 30° onto a flat and stiff sheet of nylon. 45° orthogonal fibre lays were employed for implantable sheet materials

2.12 g of commercial fibroin powder were dissolved in 60 ml of 9.5 M aqueous lithium bromide by stirring continuously at room temperature. The resulting solution was transferred to a weighed dialysis tube, the ends knotted and tube reweighed to determine the weight of solution. Dialysis was carried out against several changes of distilled water for 2-3 days at 4° C. Thereafter the dialysis tube was transferred to dry air until the weight of the fibroin solution was reduced to half of the initial wet weight of the contents of the dialysis tube before dialysis. Thus the final fibroin solution which was stored at 4° C. contained about 40% fibroin w/v. This solution was liberally painted on the fibre lay which was then allowed to dry under ambient conditions.

3. Silk/silk composites prepared in step 2 were then placed on sheet of filter paper covering 2 g of dried paraformaldehyde moistened with 0.2 ml of distilled water in a 0.5 litre jar. After sealing the jar the silk/silk composite was cross-linked by heating to 100° C. for 2 hours before cooling and washing thoroughly with warm water. The composite device after air drying is shown in FIG. 1.

### Preparation of Sutures

A suture according to an embodiment of the invention can be produced by braiding 14 brin Tussah silk fibres on a standard braining machine. The particular settings used will depend on the particular braiding machine, size of suture, etc.

### Preparation of Sheet Materials

Sheet materials can be formed by winding fibres around a plastic former, coating with a matrix and cross-linking. A simple sheet implant can be formed, for example, by winding 14 brin fibres around a plastic former at a 45° crossing angle to produce an orthogonal lay. Once wound, regenerated Bombyx mori fibroin is panted onto the fibres and allowed to dry. The material is then cross-linked using formaldehyde vapour at 85° C.

The crossing angle of winding can be selected to provide elasticity of the material in a given direction. For example, a ligament implant formed using this basic technique may have a crossing angle of 10° as opposed to 45° described above.

The foregoing is considered illustrative of the principles of the invention and since numerous modifications will occur to those skilled in the art, it is not intended to limit the invention to the exact construction and operation described. All suitable modifications and equivalents fall within the scope of the claims.

1. An implantable device comprising a structure formed from one or more silk elements made at least partially of a poly(alanine) Wild silk protein.

2. A device as claimed in claim 1, wherein the structure is resorbable when placed in a human or animal body.

3. The implantable device according to claim 1, wherein the structure has a half-resorption time of 2 weeks to 9 months.

4. A device as claimed in claim 1, wherein the silk elements are derived from the cocoons of a satumiid silk moth.

5. A device as claimed in claim 1, wherein the silk elements are formed from a solution of regenerated silk.

6. A device as claimed in claim 1, wherein the structure further comprises a peroxidase.

7. A device as claimed in claim 1, wherein the structure further comprises a silk protein matrix between the silk elements.

8. A device as claimed in claim 1, comprising a suture.

9. A device as claimed in claim 1, comprising a prosthesis.

10. A method for the manufacture of an implantable device comprising:
providing of a poly(alanine) Wild silk protein;
manufacturing silk elements from the silk protein; and
forming a structure from the silk elements to create the device.

11. A method as claimed in claim 10, wherein the step of the providing poly(alanine) Wild silk protein comprises providing native silk protein solution obtained directly from a poly(alanine) Wild silkworm.

12. A method as claimed in claim 10, wherein the step of the providing poly(alanine) Wild silk protein comprises the dissolution of a degummed silk to form a regenerated silk solution.

13. A method as claimed in claim 10, wherein the step of providing poly(alanine) Wild silk comprises providing native silk protein directly from the silk glands of a satumiid silkworm.

14. A method as claimed in claim 11, wherein the step of manufacturing silk elements comprises extrusion of the silk elements from the poly(alanine) Wild silk protein solution.

15. A method as claimed in claim 10, wherein the step of providing poly(alanine) Wild silk protein comprises unreeling silk from a cocoon of a saturniid Wild silk moth.

16. A method as claimed in claim 10, further comprising varying the degree of crystallinity of the silk element.

17. A method as claimed in claim 16, comprising exposing the silk element to one or more of formic acid, methanol, hot di- or tricarboxylic acid, and a dialdehyde.

18. A method as claimed in claim 17, wherein the tricarboxylic acid is citric acid.

19. A method as claimed in claim 17, wherein the dialdehyde is glutaraldehyde.

20. A method as claimed in claim 16, further comprising exposing the silk elements to steam and stretching them.

21. A method as claimed in claim 17, wherein the silk elements are exposed to a reducing agent after exposing them to a dialdehyde.

22. A method as claimed in claim 21, wherein the silk elements are exposed to a solution containing one or more amino acids after exposing them to a dialdehyde and before exposing them to a reducing agent.

23. A method as claimed in claim 21, wherein the reducing agent is sodium borohydride.

24. A method as claimed in claim 21, wherein the reducing agent is cyanoborohydride.

25. A method as claimed in claim 17, comprising exposing the silk element to a methanol solution of between 30% and 60% by volume.

26. A method as claimed in claim 10, further comprising binding peroxidase to the silk element.

27. A method as claimed in claim 10, wherein the structure is formed by braiding the silk elements.

28. A method as claimed in claim 10, wherein the structure is formed by winding silk elements around a former, coating with a matrix material and cross linking the matrix material.

29. A suture when produced according to a method as claimed in claim 10.

30. A prosthesis when produced according to a method as claimed in claim 10.
